(19) United States
(12) Patent Application Publication
(10) Pub. No.: US 2006/0193784 A1
(43) Pub. Date: Aug. 31, 2006

(54) Scopolamine sublingual spray for the treatment of motion sickness

(76) Inventors: Peter Crooks, Nicholasville, KY (US); Abeer Al-Ghananeem, Lexington, KY (US); Ahmad H. Malkawi, Lexington, KY (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/360,690

(22) Filed: Feb. 24, 2006

Related U.S. Application Data
(60) Provisional application No. 60/656,359, filed on Feb. 25, 2005.

(51) Int. Cl. A61K 31/46; A61L 9/04

(52) U.S. Cl. 424/45; 514/304

(57) ABSTRACT

This invention relates to a scopolamine spray for sublingual administration, used in the treatment and prevention of motion sickness, as well as the treatment and prevention of similar symptoms, such as nausea and vomiting, caused by conditions other than motion sickness. Also provided are methods of treatment, prevention and inhibition of these conditions and symptoms, as well as a metered dosage system for administration of the spray.

### FIELD OF THE INVENTION

This invention relates to a scopolamine spray for sublingual administration, used in the treatment and prevention of motion sickness, as well as the treatment and prevention of similar symptoms, such as nausea and vomiting, caused by conditions other than motion sickness. Also provided are methods of treatment, prevention and inhibition of these conditions and symptoms, as well as a metered dosage system for administration of the spray.

### BACKGROUND

Motion sickness, as well as other conditions which also cause symptoms such as nausea, are very common. Motion sickness is the nausea, vomiting, and related symptoms caused by repetitive angular and linear acceleration and deceleration. Other symptoms may include yawning, hyperventilation, salivation, pallor, profuse cold sweating, and somnolence. Aerophagia, dizziness, headache, palor, cold sweats, general discomfort, and fatigue may also occur.

Once nausea and vomiting develop, a patient may become weak and unable to concentrate. Prolonged vomiting, regardless of the cause, may lead to arterial hypotension, dehydration, inanition, and depression. Nausea and vomiting can be a serious complication in patients with other illnesses. See Merck manual, Section 20, Chapter 282.

Current treatment options, including oral formulations and transdermal patches, do not provide quick relief from these symptoms. Further, these options often have poor bioavailability and unpleasant side effects. Thus, new agents and methods of administration of the agents are needed to provide fast relief these symptoms.

### SUMMARY OF THE INVENTION

The present invention provides a scopolamine spray for sublingual administration, to be used in the treatment and prevention of motion sickness, as well as the treatment and prevention of similar symptoms, such as nausea and vomiting, caused by conditions other than motion sickness. The present invention also provides methods of treatment as well as a metered dosage system for use in administration of the scopolamine spray.

In one of its aspects, a pharmaceutical composition comprising scopolamine or a pharmaceutically acceptable salt thereof and a pharmaceutically acceptable carrier, wherein the scopolamine or pharmaceutically acceptable salt thereof is provided in a form suitable for sublingual administration is provided.

In another aspect, a liquid spray formulation, comprising (i) scopolamine or pharmaceutically acceptable salt or free base thereof, (ii) buffered water; and (iii) a polar organic solvent is provided. The polar organic solvent is present in an amount sufficient to enhance the solubility of the scopolamine free base or salt thereof in the water. The scopolamine may be present as the free base or salt. The formulation may be partially pressurized. Preferably, the scopolamine or pharmaceutically acceptable salt or free base thereof is present in the formulation at a concentration of 0.1-10 mg/ml.

Preferably, the polar organic solvent is an alcohol. The alcohol may include, but is not limited to, ethanol, propylene glycol, glycerol, polyethylene glycol and mixtures thereof. Preferably, the alcohol is ethanol. Preferably, the polar organic solvent is present in an amount of 0-90% w/w.

The formulation may be buffered. The buffer may include citrate or phosphate buffer. Preferably, the formulation has a pH of less than 5. More preferably, the formulation has a pH of about 3.5. The formulation may further comprise a sweetener. Preferably, the sweetener is mannitol, saccharin, and/or saccharin sodium. The formulation may also further comprise a flavoring agent. Preferably, the flavoring agent is menthol.

The formulation may further comprise a penetration enhancer. Preferably, the penetration enhancer is chitosan. Preferably, the formulation is suitable for sublingual administration. The formulation may further comprise a mucoadherant. The mucoadherant may include, but is not limited to chitosan, polyvinyl pyrrolidone, and/or gelatin.

In another aspect, the invention provides a liquid spray formulation, comprising (i) scopolamine or pharmaceutically acceptable salt or free base thereof, in an amount of 433.5 mg; (ii) phosphate buffer, in the amount of 100 qs; (iii alcohol in the amount of 30 mL; (iv) mannitol in the amount of 400 μg; (v) propylene glycol in the amount of 5 mL; and (vi) chitosan in the amount of 2 mg.

In yet another aspect, the invention provides a method of providing fast relief from the symptoms of motion sickness, comprising administering to a subject in need thereof a pharmaceutically effective amount of scopolamine, by spraying the scopolamine onto the subject's sublingual mucosa. The symptoms of motion sickness may include, but are not limited to, nausea, emesis, vertigo, yawning, hyperventilation, salivation, pallor, profuse cold sweating, somnolence, aerophagia, dizziness, headache, and fatigue. The scopolamine may be in the form of a scopolamine free base or hydrobromide salt, or any acceptable salt dissolved in an ethanolic solution.

In another aspect, the invention provides a method of providing fast relief from the symptoms of motion sickness comprising administering to a subject in need thereof a liquid spray formulation, comprising (i) scopolamine or pharmaceutically acceptable salt or free base thereof, (ii) buffered water; and (iii) a polar organic solvent is provided. The polar organic solvent is present in an amount sufficient to enhance the solubility of the scopolamine free base or salt thereof in the water. The formulation is sprayed onto the subject's sublingual mucosa.

In another aspect, the invention provides a metered dose dispensing system for the administration of a liquid spray formulation, which comprises (i) scopolamine or pharmaceutically acceptable salt or free base thereof, (ii) buffered water; and (iii) a polar organic solvent is provided. The polar organic solvent is present in an amount sufficient to enhance the solubility of the scopolamine free base or salt thereof in the water. The metered dose dispensing system comprises a sealed container fitted with a metering pump, an actuator and a channeling device. Preferably, the metered dose dispensing system contains a metering chamber which is adapted for dispensation with the container in the upright orientation, and wherein the metering chamber is in communication with the formulation by means of a dip-tube.

In another aspect the, the invention provides a method of providing relief from nausea any vomiting, comprising administering to a subject in need thereof a liquid spray formulation, which comprises (i) scopolamine or pharmaceutically acceptable salt or free base thereof, (ii) buffered water; and (iii) a polar organic solvent is provided. The polar organic solvent is present in an amount sufficient to enhance the solubility of the scopolamine free base or salt thereof in the water. The formulation is sprayed onto the subject's sublingual mucosa. Preferably, the relief from nausea and vomiting is achieved within 20 minutes. More preferably, the relief from nausea and vomiting is achieved within 5 minutes. The nausea and vomiting may be caused by a condition other than motion sickness.

In yet another aspect, the invention provides a method of providing relief from nausea and vomiting caused by the administration of a medicament, comprising administration of a liquid spray formulation, which comprises (i) scopolamine or pharmaceutically acceptable salt or free base thereof, (ii) buffered water; and (iii) a polar organic solvent is provided. The polar organic solvent is present in an amount sufficient to enhance the solubility of the scopolamine free base or salt thereof in the water. The formulation may be administered before, concurrently, or after the administration of the medicament. The medicament may be an anti-cancer drug or an anti-viral drug.

### BRIEF DESCRIPTION OF THE FIGURES

FIG. 1 illustrates the mean plasma scopolamine levels following sublingual spray dosing and intravenous (IV) administration at 100 μg scopolamine equivalent dose (n=3), as seen in Example 1. All values show the mean±SEM.

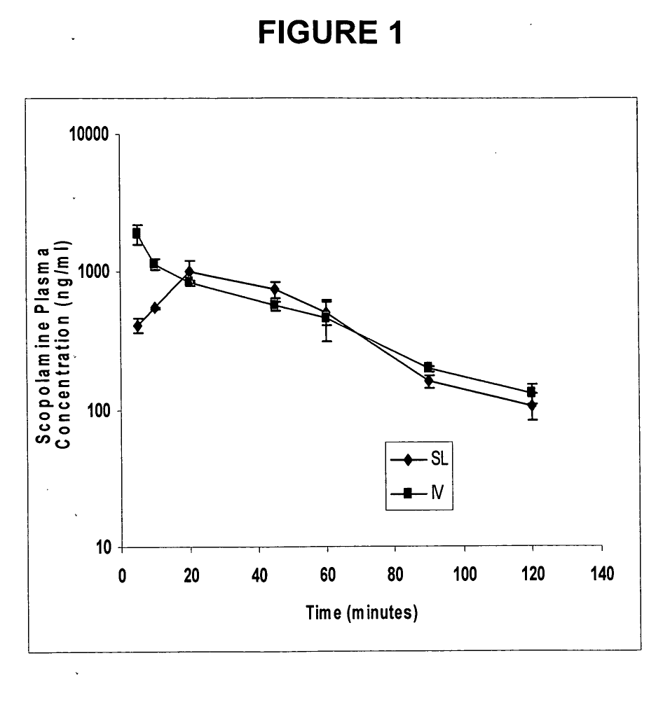

### DETAILED DESCRIPTION OF THE INVENTION

As described above, this invention provides a scopolamine spray for sublingual administration, used in the treatment and prevention of motion sickness and symptoms caused by other conditions, such as nausea and vomiting. Also provided are methods of treatment, prevention and inhibition of these conditions and symptoms, as well as a metered dosage system for administration of the spray.

However, prior to describing this invention in further detail, the following terms will first be defined.

Definitions

In accordance with this detailed description, the following abbreviations and definitions apply. It must be noted that as used herein, the singular forms “a”, “an”, and “the” include plural referents unless the context clearly dictates otherwise. Thus, for example, reference to “compounds” includes a plurality of such compounds and reference to “the dosage” includes reference to one or more dosages and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Unless otherwise stated, the following terms used in the specification and claims have the meanings given below:

“Pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and claims includes both one and more than one such carrier.

“Treating” or “treatment” of a disease includes:

(1) preventing the disease, i.e., causing the clinical symptoms of the disease not to develop in a mammal that may be exposed to or predisposed to the disease but does not yet experience or display symptoms of the disease,

(2) inhibiting the disease, i.e., arresting or reducing the development of the disease or its clinical symptoms, or

(3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Pharmaceutically acceptable salt” refers to pharmaceutically acceptable salts of scopolamine which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

“Optional” or “optionally” means that the subsequently described event or circumstance may, but need not, occur, and that the description includes instances where the event or circumstance occurs and instances in which it does not.

The term “fast” refers to the speed at which the present compositions and formulations provide relief from motion sickness and similar conditions. The term “fast” encompasses immediate relief up to about one hour, from the time the composition or formulation is administered.

The term “suitable for sublingual administration” refers to any mode of administration of a medicament to the tissue under the tongue. For example, a spray may be used.

The term “subject in need thereof” refers to any animal in need of relief from the symptoms of motion sickness, or the same or similar symptoms caused by any other disease or condition. Preferably, the subject is a mammal. More preferably, the subject is human.

Scopolamine [L-(−)-hyoscine] has been the drug of choice for the symptomatic treatment of motion sickness for many years (Holling et al., Prevention of seasickness by drugs. Lancet 1944, 127-129; and Money, Motion sickness. Physiol. Rev. 1970, 50, 1-39).

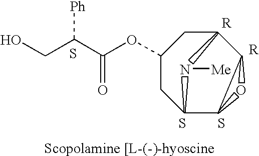

Scopolamine is a belladonna alkaloid. Scopolamine competitively inhibits the muscarinic receptors for acetylcholine, and acts as a nonselective muscarinic antagonist, producing peripheral antimuscarinic properties and central sedative, antiemetic, and amnestic effects (Ali-Melkkila et al., Pharmacokinetics of anticholinergic drugs. Acta Anaesthesiol Scand. 1993, 37, 633-642.). Currently, four dosage forms for the administration of scopolamine are commonly used. These include parenteral injection, ophthalmic solution, oral tablets, and skin patches. Tablets and skin patches are used primarily for the prevention of motion sickness.

The intravenous (IV) route has shown one hundred percent bioavailability. However, the invasive nature of the IV procedure, and the difficulty of administration, makes IV administration unfeasible for the treatment of everyday nausea and other symptoms. Further, occasional technical constraints, such as those experienced during driving or space flights, limit the usefulness of this route of administration.

The variability in absorption and poor bioavailability (for example, 10.7-48.2% bioavailability) seen with oral administration indicate that the oral route is neither reliable nor effective for this scopolamine (Putcha et al., Pharmacokinetics and oral bioavailability of scopolamine in normal subjects. Pharm. Res. 1989, 6(6), 481-485). The transdermal administration of scopolamine also has limitations. The plasma concentrations of the drug indicate major interindividual variations (Renner et al., Pharmacokinetics and pharmacodynamics in clinical use of scopolamine. Therapeutic drug monitoring. 2005, 27(5), 655-665). Moreover, the transdermal patch releases 0.5 mg alkaloid over a relatively long period of 72 hours, and scopolamine concentrations in plasma declined more slowly after the patches were removed relative to after an IV dose (Shaw et al., Programmed systemic delivery by the transdermal route. Trends Pharmacol. Sci. 1980, 1(8), 208-211).

While oral or transdermal systems may be readily provided to deliver scopolamine to a person experiencing or seeking to prevent nausea and other symptoms, there is a delay of onset of action before the effective entry of the scopolamine into the patient circulation. For example, the peak plasma concentration is not reached until 12-16 hours after transdermal dosing (Cintron et al., A sensitive radioreceptor assay for determining scopolamine concentration in plasma. J. Pharm. Sci. 1987, 76, 328-332; and Shaw, et al., J. Programmed systemic delivery by the transdermal route. Trends Pharmacol. Sci. 1980, 1(8), 208-211), and 30 minutes after oral administration (Renner et al., Pharmacokinetics and pharmacodynamics in clinical use of scopolamine. Therapeutic drug monitoring. 2005, 27(5), 655-665.). In the case of sudden air or water turbulence, a person may immediately need treatment for motion sickness that they did not anticipate. The delay in the drug reaching the circulation after patch application or oral administration means that one must anticipate that they may experience motion sickness, up to 16 hours prior to the sickness.

This delay, coupled with the side effects associated with the patch and oral administration, including dry mouth, dizziness, blurred vision, confusion, and hallucinations, show that there is a need for an alternative method of administration of scopolamine.

The present invention is directed to the administration of scopolamine by the sublingual route. Sublingual administration (physiological pH ˜6.5 as in Quintanar-Guerrero et al., In vitro evaluation of the bioadhesive properties of hydrophobic polybasic gels containing N,N-dimethylaminoethyl methacrylate-co-methyl methacrylate. Biomaterials. 2001, 22, 957-961) has the potential for providing an alternative to intravenous and oral dosing for rapid delivery of drugs to the systemic circulation. It was found that only 2.6% of non-metabolized scopolamine is excreted in urine, which suggests a first-pass metabolism after oral administration of scopolamine (Renner et al., Pharmacokinetics and pharmacodynamics in clinical use of scopolamine. Therapeutic drug monitoring. 2005, 27(5), 655-665). Thus, sublingual drug delivery by-passes gastrointestinal and hepatic pre-systemic elimination, and is a useful form of drug delivery for patients with swallowing problems. This is especially effective if a patient is experiencing nausea and emesis, and cannot readily swallow an oral dosage form.

Scopolamine Spray

The present invention provides a pharmaceutical composition comprising scopolamine, provided for sublingual administration. The present invention also provides a liquid formulation for administering scopolamine to the sublingual mucosa (i.e., under the tongue) by a spray. This formulation preferably comprises scopolamine or acceptable salt or free base thereof, buffered water, and a polar organic solvent.

In general, the compounds of the subject invention will be administered in a therapeutically effective amount by any accepted sublingual modes of administration. Preferably, the formulation is administered as a liquid spray composition. Such compositions are prepared in a manner well known in the pharmaceutical art. Preferably, the spray is administered directly to the sublingual mucosa.

The scopolamine is present in the compositions and formulations in an amount sufficient to prevent, treat, relieve, and/or inhibit motion sickness and other conditions with similar symptoms of motion sickness. The active compound is effective over a wide dosage range and is generally administered in a pharmaceutically or therapeutically effective amount. The therapeutic dosage of the compounds of the present invention will vary according to, for example, the particular use for which the treatment is made, the manner of administration of the compound, the health and condition of the patient, and the judgment of the prescribing physician. For sublingual administration by spray, the dose will typically be in the range of about 0.1 mg/ml to about 10 mg/ml, and preferably about 0.5 mg/ml to about 5 mg/ml. Effective doses can be extrapolated from dose-response curves derived from in vitro or animal model test systems.

The actual amount of the compound, i.e., scopolamine and acceptable salts and free bases thereof, will depend on a number of factors, such as the severity of the disease, i.e., the condition or disease and symptoms, the age and relative health of the subject, the potency of the compound used the route and form of administration, and other factors.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture assays and animal studies can be used in future formulating of a range of dosage for use in humans and other animal patients. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range which includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The amount administered to the patient will vary depending upon what is being administered, the purpose of the administration, such as prophylaxis versus therapy, the state of the patient, the manner of administration, and the like. In therapeutic applications, compositions are administered to a patient already suffering from symptoms and/or a condition in an amount sufficient to cure or at least partially arrest the symptoms and complications. An amount adequate to accomplish this is defined as “therapeutically effective dose.” Amounts effective for this use will depend on the age, weight and general condition of the subject/patient, and the like.

The polar organic solvent is preferably present in an amount which will enhance the solubility of the scopolamine in water. Preferred organic solvents include, but are not limited to, alcohols, such as ethanol, propylene glycol, glycerol, polyethylene glycol and mixtures thereof. The polar organic solvent may be present in the formulation in an amount of about 0-90% w/w. The formulation may be buffered, as appropriate.

The compositions administered to a subject are in the form of pharmaceutical compositions. These compositions may be sterilized by conventional sterilization techniques, or may be sterile filtered. It will be understood that use of certain of the foregoing excipients, carriers, or stabilizers will result in the formation of pharmaceutical salts. When employed as pharmaceuticals, the compounds of the subject invention are usually administered in the form of pharmaceutical compositions. This invention also includes pharmaceutical compositions, which contain as the active ingredient, one or more of the compounds of the subject invention above, associated with one or more pharmaceutically acceptable carriers or excipients. The excipient employed is typically one suitable for administration to human subjects or other mammals. In making the compositions of this invention, the active ingredient is usually mixed with an excipient, diluted by an excipient. When the excipient serves as a diluent, it can be a solid, semi-solid, or liquid material, which acts as a vehicle, carrier or medium for the active ingredient.

The compositions of the invention can be formulated so as to provide quick, sustained or delayed release of the active ingredient after administration to the patient by employing procedures known in the art. The scopolamine formulation may further comprise a sweetened, such a mannitol, saccharin, and saccharin sodium. The formulation may further comprise a flavoring agent, such as menthol.

To assist in the speed of efficacy and bioavailability, the formulation may also comprise a penetration enhancer. Preferably, the penetration enhancer is chitosan. When formulated with a penetration enhancer such as chitosan, the bioavailabliliy of the formulation can reach 90% or greater. The formulation may also comprise a mucoadherant to increase the residence time on the mucosa; including chitosan, polyvinyl pyrrolidone, or gelatin.

The formulation may further comprise a moisturizing agent, such as propylene glycol, or polyethylene glycol. The formulation may further comprise a preservative such as sodium metabisulphite, benzalkonium chloride, or ethanol. The formulation may further comprise an antioxidant, such as butylated hydroxyltoluene, ascorbic acid, alkyl gallates, or tocopherols. The formulation may further comprise an ionic or nonionic surfactant, such as sodium lauryl sulfate, or sorbitan esters.

The liquid forms in which the compositions of the present invention may be incorporated for administration by spray include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles. A spray formulation may be prepared by methods well known in the art.

According to one aspect of the invention, the compound may be administered alone, or in combination with any other medicament which causes side effects such as nausea, vertigo, and vomiting/emesis. Such medicaments include, but are not limited to, chemotherapeutic agents, anti-virals, and other anti-HIV drugs.

When administered in combination, the compounds may be administered in the same formulation as these other compounds or compositions, or in a separate formulation. When administered in combination, the scopolamine spray may be administered prior to, following, or concurrently with the other compounds and compositions.

When administered as a spray for sublingual administration, the scopolamine is preferably in the form of scopolamine free base or hydrobromide salt, or any acceptable salt dissolved in an ethanolic solution.

Suitable methods and formulations for use in the present invention are found in REMINGTON'S PHARMACEUTICAL SCIENCES, Mace Publishing Company, Philadelphia, Pa., 17th ed. (1985).

Methods of Treatment and Prevention

The present invention provides methods of treating, inhibiting and/or preventing motion sickness, symptoms associated with motion sickness, and other conditions exhibiting any symptoms also seen in motion sickness. The present invention may also be used to treat and prevent any instances of such symptoms, even if not associated with motion sickness. Such conditions and symptoms for treatment using the scopolamine of the present invention include nausea, vomiting (emesis), any symptom caused by repetitive angular and linear acceleration and deceleration, yawning, hyperventilation, salivation, pallor, profuse cold sweating, aerophagia, somnolence, dizziness, headache, general discomfort, and fatigue. The present invention may be used to treat nausea, vertigo, and vomiting caused by pregnancy, chemotherapeutic treatments, radiation treatments, gastroenteritis, migraines, and any other condition or disease causing these symptoms.

The formulation is preferably administered as a spray. Preferably, the spray is administered directly to the sublingual mucosa, i.e., the formulation is sprayed directly onto the tissue under the patient's tongue. By administering the scopolamine directly to the sublingual mucosa, the patient can experience fast and even immediate relief, while still maintaining a high level of bioavailability. A patient suffering from these symptoms can feel relief within 1-5 minutes, with a maximum concentration of the drug being reached within 20 minutes or faster. Thus, someone suffering from motion sickness and/or symptoms such as nausea and emesis no longer has to anticipate the symptoms by taking a dosage form hours prior to the anticipated sickness. Further, a subject can now quickly treat and even prevent discomfort and sickness caused by an unexpected event.

Metered Dosage System

The present invention further provides a device and system for administering the scopolamine spray. Such a system can include a metered dose dispensing system, providing a convenient way to confirm that each spray dose is identical in amount. The metered dosage system may comprise a sealed container, which is fitted with a metering pump, an actuator and a channeling device. The metered dosage system may further contain a metering chamber adapted for dispensation with the container in the upright orientation. The metering chamber would be placed in communication with the formulation by means of a dip-tube.

Metered dosage systems well known in the art may be used. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The following example is offered to illustrate this invention and is not to be construed in any way as limiting the scope of this invention.

### EXAMPLES

### Example 1

A study was performed to develop a sublingual spray drug delivery formulation of scopolamine (L-(−)-hyoscine), and then to evaluate the absolute bioavailability of scopolamine following sublingual delivery.

Rabbits received a single scopolamine equivalent spray dose of 100 μg/kg (about 300 μg/rabbit), and the results were compared to intravenous administration of the drug. Blood samples were collected at different time points, and plasma scopolamine concentrations were determined utilizing a new, sensitive, and specific LC/MS method of analysis with electrospray ionization detection. Considering the limitations of delivering scopolamine orally or transdermally to patients undergoing motion sickness, the sublingual route using a spray delivery dosage form was found to be a highly useful alternative modality to prevent nausea and vomiting associated with motion sickness.

Scopolamine has a weak basic character (pKa=7.6) and a reasonable lipid solubility with a partition coefficient equal to 1.2 (Renner et al., Pharmacokinetics and pharmacodynamics in clinical use of scopolamine. Therapeutic drug monitoring. 2005, 27(5), 655-665). Thus, the effect of chitosan as an absorption enhancer was also tested.

Scopolamine hydrobromide trihydrate, ethanol, propylene glycol, mannitol, and sodium phosphate, were obtained from Sigma-Aldrich Chemical Co. (St Louis, USA). Hydrochloric acid, purified water USP, ammonium acetate, methanol, chloroform, and HPLC grade acetonitrile were obtained from Fisher Scientific (Pittsburgh, Pa.). Water for HPLC use was passed through a reverse osmosis system (Milli-Q® Reagent Water System) before use. Isoflourane gas for anesthesia was provided by VMC Anesthesia (Ohmeda Waukesha, Wis.). Siliconized microcentrifuge tubes, vials, and tips were purchased from Fisher Scientific (Fair Lawn, N.J.). Saline (0.9%, injectable) was purchased from Baxter Healthcare Corporation (Deerfield, Ill.). Heparinized caraway capillary tubes were purchased from Baxter Healthcare Corporation (McGraw Park, Ill.). Tuberculin Slip tip Sterile Catheters were purchased from J&J Medical (New Brunswick, N.J.).

Male New Zealand albino rabbits weighing between 3.0-3.5 kg (Myrtle's Rabbitry Inc., Thompson Station, Tenn.) were used. The animal work was conducted at the University of Kentucky Chandler Medical Center, Division of Laboratory Animal Resources (DLAR). All research and testing activities related to this work were reviewed and approved by the Institutional Animal Care and Use Committee (IACUC) prior to the initiation of this research, and during its execution.

The formulations were prepared as in Table 1.

TABLE 1
Active and inactive contents of scopolamine sublingual spray
formulation solutions 1 and 2.

| Material | Formulation 1 | | Formulation 2 | |
|---|---|---|---|---|
| Scopolamine HBr.3H₂O | 433.5 | mg | 433.5 | mg |
| Absolute alcohol | 30 | ml | 30 | ml |
| Mannitol | 400 | μg | 400 | μg |
| Propylene glycol | 5 | ml | 5 | ml |
| Chitosan | — | | 2 | mg |
| Phosphate buffer (0.5 M, pH 3.5) | 100.0 | qs | 100.0 | qs |

In Vivo Sublingual and Intravenous Studies

Following introduction of anesthesia (isoflourane general anesthetic gas), a catheter was placed in the marginal ear vein of the rabbit for blood sample collection. For sublingual spray administration, a scopolamine dose 100 μg/kg of formulation solution 1 was applied to the sublingual mucosa of the rabbit through a spray bottle (n=3 rabbits/per route).

TABLE 2
Area under the curve and absolute bioavailability of scopolamine
sublingual spray formulation 1 in rabbits (n = 3)

| | | AUC_{∞} | Absolute |
|---|---|---|---|
| | | (ng · min/mL) | bioavailability (%) |
| | Route | mean ± S.E. | mean ± S.E. |
| Dose | Intravenous | 76512.8 ± 10273 | 100 |
| 300 mg | Sublingual | 61067.6 ± 9605 | 80 ± 2.7% |
| scopolamine | Formulation 1 | | |
| equivalent | Formulation 2 | | |

A separate in vitro spray weight evaluation was performed for the spray bottle before dosing. The spray bottle was hand-filled with 2.5 mL deionized water and actuated ten times for priming before obtaining spray weight data. After priming, net spray weight measurements were taken for ten consecutive actuations. Target delivery weight for each single spray was about 0.1 g. For intravenous administration, scopolamine hydrobromide aqueous solution was utilized. A sterile drug solution was prepared by filtration (double 0.22 μm filters), and a dose of 100 μg/kg scopolamine was injected into the marginal ear vein cannula followed by a 0.1 mL flush with 10% (v/v) heparin/normal saline solution to keep the cannula patent.

Aliquot parts of 1 mL blood samples were collected at baseline, before scopolamine dose administration; immediately after scopolamine administration; and subsequently at 5, 10, 20, 45, 60, and 120 minutes following scopolamine administration. Blood samples were injected into pre-heparinized tubes and immediately placed on ice. Plasma was separated by centrifugation at 3000 rpm for 10 min, placed in polypropylene tubes, and frozen at −20° C. until the time of analysis.

Sample Preparation

Chlorobutane (1 mL) was added to 500 μL plasma in 2 mL polypropylene test tubes. The samples were vortexed for 60 seconds and centrifuged at 8,000 rpm for 10 minutes. 800 μL aliquot parts of the resulting supernatant were directly transferred to autosampler vials, evaporated to dryness with nitrogen gas at ambient temperature, and then reconstituted with 100 μL methanol. 25 μl aliquot parts of this final solution were injected onto the HPLC-MS system.

HPLC-MS Analysis

Chromatography was performed on a Waters Sunfire C₁₈ (4.6 mm×250 mm, 5 μm) column with a mobile phase consisting of 30% ammonium acetate (10 mM, pH 4), 40% methanol, and 30% acetonitrile. The flow-rate was set at 0.3 mL/min. The LC-MS system consisted of a Waters 2690 HPLC pump (Waters, Milford, Mass.), a Waters 2695 autosampler, and a Micromass ZQ detector (Waters, Milford, Mass.) which utilized electrospray ionization (ESI). Selected ion monitoring (SIM) was performed in the positive mode for scopolamine, M+=304 m/z (dwell time 0.8 s), the capillary voltage was 3.30 kV and the cone voltage was 32 V. The source block and desolvation temperatures were 100 and 300° C., respectively. Nitrogen was used as the nebulization and drying gas at flow rates of 70 and 450 L/h, respectively. Calibration curves were constructed using a linear regression of the drug peak area versus nominal drug concentrations. The method was validated over the concentration range used, and found to be satisfactory for the determination of scopolamine in rabbit plasma over the concentration range of 10-2000 ng/ml. The limit of quantification (LOQ) was established at 10 ng/ml. MS control and spectral processing were performed using MassLynx™ software, version 3.5.

Pharmacokinetic Analysis

Concentration-time profiles of scopolamine after IV and sublingual administration of formulations 1 and 2 (See Table 1) were evaluated by a non-compartmental model (WinNonlin Professional, version 4.1, Pharsight Corporation, Mountain view, Calif.). The pharmacokinetic parameters, such as terminal elimination half life (t_{1/2}), area under the curve from 0 to infinity, AUC_{0-∞} were estimated using this software.

After a single dose, maximum plasma concentration (Cₘₐₓ), and time to reach maximum concentration (Tₘₐₓ) were also determined. The absolute bioavailability of the sublingual formulation was calculated by the equation below:

$$F = \frac{AUC SL}{AUC IV} ⨯ \frac{Dose IV}{Dose SL} ⨯ 100$$

where F is the percent absolute bioavailability, and AUC_{SL}, AUC_{IV}, Dose_{IV}, Dose_{SL} are the area under the curve and corresponding dose for the sublingual and intravenous administrations, respectively.

Following sublingual spray dose, the average Cₘₐₓ was 1024.4±177 ng/ml, and AUC value was found to be 61067.6±9605 ng.min/ml. Relative to the 100% bioavailability from the intravenous route, the bioavailability was 80±2.7% after sublingual spray administration. Pharmacokinetic parameters are shown in Table 3.

TABLE 3
Pharmacokinetic parameters following sublingual spray administration
of formulation 1 and formulation 2 in rabbits (n = 3)

| Parameter | Formulation 1 | Formulation 2 | intravenous |
|---|---|---|---|
| Cₘₐₓ (ng/mL) | 1024.4 ± 177 | | N/A |
| tₘₐₓ (min) | 20 | | N/A |
| t_{1/2} (min) | 27.9 | | 34 |

While the present invention has been described with reference to specific embodiments, this application is intended to cover those various changes and substitutions that may be made by those of ordinary skill in the art without departing from the spirit and scope of the appended claims.

What is claimed is:

1. A pharmaceutical composition comprising scopolamine or a pharmaceutically acceptable salt thereof and a pharmaceutically acceptable carrier, wherein the scopolamine or pharmaceutically acceptable salt thereof is provided in a form suitable for sublingual administration.

2. A liquid spray formulation, comprising:
(i) scopolamine or pharmaceutically acceptable salt or free base thereof,
(ii) buffered water; and
(iii) a polar organic solvent,
wherein the said polar organic solvent is present in an amount sufficient to enhance the solubility of the scopolamine free base or salt thereof in the water.

3. The formulation of claim 2, wherein the scopolamine is present as the free base or salt.

4. The formulation of claim 2, wherein the formulation is partially pressurized.

5. The formulation of claim 2, wherein the scopolamine or pharmaceutically acceptable salt or free base thereof, is present at a concentration of 0.1-10 mg/ml.

6. The formulation of claim 2, wherein the polar organic solvent is an alcohol.

7. The formulation of claim 6, wherein the alcohol is selected from the group consisting of ethanol, propylene glycol, glycerol, polyethylene glycol and mixtures thereof.

8. The formulation of claim 7, wherein the alcohol is ethanol.

9. The formulation of claim 2, wherein the polar organic solvent is present in an amount of 0-90% w/w.

10. The formulation of claim 2, wherein the formulation is buffered.

11. The formulation of claim 10, wherein the formulation is buffered with citrate or phosphate buffer.

12. The formulation of claim 2, wherein the formulation has pH of less than 5.

13. The formulation of claim 12, wherein the formulation has a pH of about 3.5.

14. The formulation of claim 2, further comprising a sweetener.

15. The formulation of claim 14, wherein the sweetener is mannitol.

16. The formulation of claim 14, wherein the sweetener is saccharin or saccharin sodium.

17. The formulation of claim 2, further comprising a flavoring agent.

18. The formulation of claim 17, wherein the flavoring agent is menthol.

19. The formulation of claim 2, further comprising a penetration enhancer.

20. The formulation of claim 19, wherein the penetration enhancer is chitosan.

21. The formulation of claim 2, further comprising a mucoadherant.

22. The formulation of claim 21, wherein the mucoadherant is selected from the group consisting of chitosan, polyvinyl pyrrolidone, and gelatin.

23. The formulation of claim 2, wherein the formulation is suitable for sublingual administration.

24. A liquid spray formulation, comprising:
(i) scopolamine or pharmaceutically acceptable salt or free base thereof, in an amount of 433.5 mg;
(ii) phosphate buffer, in the amount of 100 qs;
(iii) alcohol in the amount of 30 mL;
(iv) mannitol in the amount of 400 μg;
(v) propylene glycol in the amount of 5 mL; and
(vi) chitosan in the amount of 2 mg.

25. A method of providing fast relief from the symptoms of motion sickness, comprising administering to a subject in need thereof a pharmaceutically effective amount of scopolamine, by spraying the scopolamine onto the subject's sublingual mucosa.

26. The method of claim 24, wherein the symptoms of motion sickness are selected from the group consisting of nausea, emesis, vertigo, yawning, hyperventilation, salivation, pallor, profuse cold sweating, somnolence, aerophagia, dizziness, headache, and fatigue.

27. The method of claim 24, wherein the scopolamine is in the form of scopolamine free base or hydrobromide salt, or any acceptable salt dissolved in an ethanolic solution.

28. A method of providing fast relief from the symptoms of motion sickness comprising administering to a subject in need thereof the formulation of claim 2, by spraying the formulation onto the subject's sublingual mucosa.

29. A metered dose dispensing system for the administration of the formulation of claim 2, comprising a sealed container fitted with a metering pump, an actuator and a channeling device.

30. The metered dose dispensing system of claim 27, containing a metering chamber which is adapted for dispensation with the container in the upright orientation, and wherein the metering chamber is in communication with the formulation by means of a dip-tube.

31. A method of providing relief from nausea any vomiting, comprising administering to a subject in need thereof the formulation of claim 2, by spraying the formulation onto the subject's sublingual mucosa.

32. The method of claim 30, wherein relief from nausea and vomiting is achieved within 20 minutes.

33. The method of claim 30, wherein relief from nausea and vomiting is achieved within 5 minutes.

34. The method of claim 31, wherein the nausea and vomiting are caused by a condition other than motion sickness.

35. A method of providing relief from nausea and vomiting caused by the administration of a medicament, comprising administration of the formulation of claim 2 before, concurrently, or after the administration of the medicament.

36. The method of claim 35, wherein the medicament is an anti-cancer drug or an anti-viral drug.
